(19) United States
(12) Patent Application Publication
(10) Pub. No.: US 2004/0175331 A1
(43) Pub. Date: Sep. 9, 2004

(54) Application of a bioactive agent to a delivery substrate

(76) Inventors: Iddys D. Figueroa, Dorado, PR (US); Vanessa I. Chinea, Aguadilla, PR (US); Orlando Ruiz, Aguadilla, PR (US); Douglas A. Sexton, La Jolla, CA (US); Winthrop D. Childers, San Diego, CA (US); James W. Ayres, Corvallis, OR (US); John Stephen Dunfield, Corvallis, OR (US)

Correspondence Address: HEWLETT-PACKARD COMPANY, Intellectual Property Administration, P. O. Box 2742400, Fort Collins, CO, 80527-2400, US

(21) Appl. No.: 10/801,380

(22) Filed: Mar. 15, 2004

Related U.S. Application Data
(63) Continuation-in-part of application No. 10/027,611, filed on Oct. 24, 2001, now Pat. No. 6,702,894.
(63) Continuation-in-part of application No. 10/028,450, filed on Oct. 24, 2001.
(63) Continuation-in-part of application No. 10/625,813, filed on Jul. 22, 2003.
(62) Division of application No. 09/877,896, filed on Jun. 7, 2001, now Pat. No. 6,623,785.

(51) Int. Cl. A61L 9/04

(52) U.S. Cl. 424/45

(57) ABSTRACT

A method of controlling a dissolution rate of a bioactive agent includes selecting a desired dot size corresponding to a target dissolution rate, and applying a bioactive agent to a delivery substrate in drops of solution configured to form dots having the desired dot size on the delivery substrate.

### CROSS-REFERENCES

This application is a continuation-in-part of U.S. patent application Ser. Nos. 10/027,611 and 10/028,450, both filed on Oct. 24, 2001, and Ser. No. 10/625,813, which was filed on Jul. 22, 2003, and is a divisional of U.S. Pat. No. 6,623,785, filed Jun. 7, 2001. The contents of the above identified applications and patent are incorporated by reference.

### BACKGROUND

Oral administration of pharmaceuticals is one of the most widely used methods of providing therapy to treat a variety of illnesses. Many medications are orally administered to a person in a dosage form such as a tablet, capsule, or liquid. Such medications can be administered buccally, sublingually, or swallowed for release into the digestive tract. Most pharmaceuticals involve dosage units in the microgram to milligram range of the purified active ingredient or ingredients, and many pharmaceuticals are made in formulations of a predetermined quantity for each tablet or capsule. Such pharmaceutical doses are frequently available in fixed strengths, such as 50 mg, 100 mg, etc.

In order to effectively handle and dispense such small dosage units, typical methods for manufacturing include mixing a known amount of the active ingredient into various solid and/or liquid substances commonly referred to as excipients or diluents. In addition, other materials such as binders, lubricants, disintegrants, stabilizers, buffers, preservatives, etc. can also be mixed with the active ingredient. Although these materials may be therapeutically inert, non-toxic, and play an important role in the manufacture of pharmaceuticals, their use nonetheless can present problems. For example, the use of diluents typically involves sequential dilutions, each of which can increase uncontrolled variability of the concentration of the active ingredient. In addition, thorough mixing requires complicated routines and expensive equipment to produce uniform doses. Known processing methods can expose the ingredients to excessive heat for durations that can be destructive to certain active ingredients. Hot spots in the mixing equipment can also contribute to variability in the doses produced. Thus, the mixing equipment may need to be cooled or production slowed to prevent excessive heat. Tight control over the various dilutions, mixings, and equipment settings are required to maintain adequate control over the accuracy and precision of the doses.

Therapeutically inactive materials must be evaluated before use to determine potential incompatibilities with the active ingredients. For example, some of these materials, such as lubricants or disintegrants, may present problems concerning the bioavailability of the active ingredient. The certification of new drugs is a lengthy and costly process, which involves animal studies and chemical trials designed to establish both the efficacy and safety of the new drug. Because a pharmaceutical's characteristics may be affected by changes in manufacturing and/or packaging, the approval process limits the approval to a particular manufacturing and packaging process. Thus, the ability to rapidly and easily change attributes of the dosage form is extremely limited in conventional pharmaceutical manufacturing systems and processes.

Drugs with a narrow therapeutic range must be precisely dosed in order to be safe and effective. If a recipient takes less than the effective dose, the desired effect will likely not occur. On the other hand, if the recipient takes more than the effective dose, the risk of toxic effects increases. Dose control for high potency drugs is frequently an issue when making solid dosage forms. Small amounts of material must be mixed homogeneously with large amounts of excipients. These mixing and subsequent dosage formation processes can yield doses that are greater than 15% above or below the intended label claim dosage and have pill to pill dosage variations greater than 6% relative standard deviation. This can be insufficient for drugs with a narrow therapeutic range. Such label claim deviation and pill to pill inconsistency can lead to drugs that do not meet standards set forth by organizations such as the United States Pharmacopeia. The many FDA generic formulation rejections and recalls for pharmaceuticals that have too high or low of a drug level are evidence that accuracy and precision are still challenges in conventional pharmaceutical manufacturing processes.

The ability to customize the release profile of a pharmaceutical can be advantageous. For example, if an active ingredient can be released so that the concentration of the active ingredient remains within a therapeutic range in a recipient's body over a 24 hour period, the recipient need only take the pharmaceutical once every day. As another example, some pharmaceuticals may be most effective when almost instantaneously absorbed by the recipient. Therefore, increasing the dissolution rate of the active ingredient can improve efficacy of the pharmaceutical. Traditional dosage forms and manufacturing techniques are characterized by limited control of the dissolution rates of the active ingredients when the dosage form is taken by a recipient. Therefore, controlling the release profiles of such drugs is difficult. Furthermore, fast release profiles associated with high dissolution rates are difficult to achieve.

Prior attempts to increase drug dissolution rates have relied on physically grinding a drug to yield micron size and smaller particles. This can cause degradation of the drug by shear and heat stress. Furthermore, particles less than 5 microns tend to agglomerate, which counters the benefits of micronization. Although agglomeration can be limited by creating liquid suspensions or emulsions, such liquids can have poor storage life and traditional methods for administering such liquids are disfavored. Soft elastic gelatin capsules can be used to keep the drug in solution, but these liquid forms can suffer from accelerated thermal degradation relative to solid state formulations.

Spray-drying and freeze-drying have also been used to generate small particles in an attempt to increase drug dissolution rates. However, agglomeration remains a problem. Another approach relies on the dissolution of the drug in organic solvents and subsequent precipitation by the addition of water or some other miscible solvent in which the drug is less soluble. However, it is frequently difficult or impossible to produce small particles with this method. Yet another alternative is to increase the dissolution rate of the drug by complexing the active drug entity with inclusion agents like cyclodextrins. For this to work the drug molecule must be amenable to inclusion into the cyclodextrin ring. Even then, the drug-cyclodextrin complex must be extensively tested for safety, which can be time consuming and expensive.

### BRIEF DESCRIPTION OF DRAWINGS

FIG. 1 schematically shows an exemplary system configured to apply a bioactive agent to a delivery substrate.

FIG. 2 schematically shows an exemplary dosage form including a delivery substrate and an applied bioactive agent.

FIG. 3 schematically shows an exemplary sheet including plural dosage forms.

FIG. 4 schematically shows a portion of an exemplary depositing subsystem configured to eject a solution including a bioactive agent onto a delivery substrate.

FIGS. 5 and 6 show an exemplary drop of solution applied to an exemplary delivery substrate.

FIG. 7 schematically shows exemplary dots of bioactive agent having different geometric surface areas.

FIG. 8 schematically shows exemplary dots of bioactive agent having different dot patterns.

FIG. 9 schematically shows exemplary dots of bioactive agent having different topographic surface areas.

FIGS. 10 and 11 are flowcharts showing methods of controlling a dissolution rate of a bioactive agent.

### DETAILED DESCRIPTION

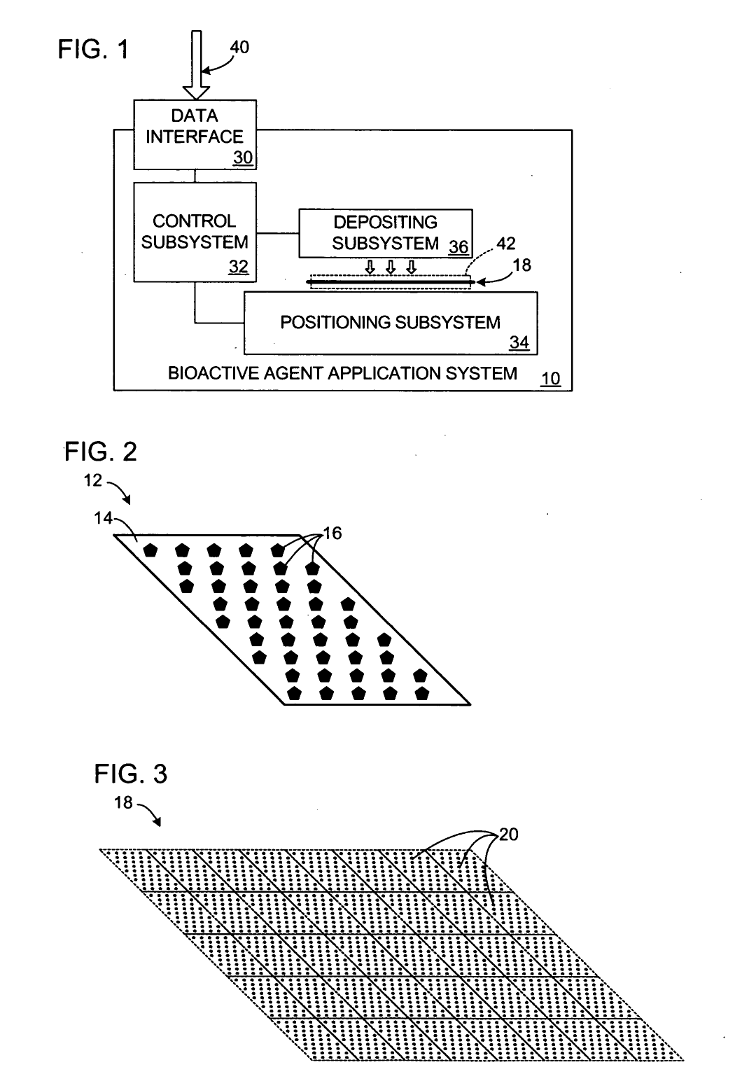

FIG. 1 schematically shows a system 10 adapted to apply a bioactive agent to a delivery substrate. For purposes of this description, the term “bioactive agent” is used to describe a composition that affects a biological function of an animal, such as a human. A nonlimiting example of a bioactive agent is a pharmaceutical substance, such as a drug, which is given to alter a physiological condition of the animal. A bioactive agent may be any type of drug, medication, medicament, vitamin, nutritional supplement, or other composition that can affect the animal.

In order for a bioactive agent to achieve its desired result, it typically must be delivered to a biological site of interest. The vast majority of drugs in use today are solid ingestibles. In order for these drugs to be absorbed into the bloodstream and transported to a biological site of interest, they usually must first be dissolved and then permeate the intestinal walls. The drugs must also avoid first pass metabolism, which occurs when the drugs are removed from the bloodstream as they pass through the liver.

Modern high throughput screening and combinatorial chemistry drug discovery methods are producing high potency drugs with high specificity. As affinities for targeted cell sites increase, the lipophilicity of the compounds tends to increase. Conversely, the aqueous solubility of the compounds tends to decrease. A decrease in the aqueous solubility of a compound typically results in a corresponding decrease in the dissolution rate of the compound. A drug with a low dissolution rate can pass through the digestive system without being absorbed in therapeutic quantities. Therefore, methods of delivering bioactive agents with high dissolution rates are desired. Drug candidates are frequently chemically modified to enhance their specificity, permeability, solubility, and dissolution rate. Trade-offs between these desired factors are made as the drug candidates are refined. Obviously, methods which can be used to enhance one or more of these desired properties without negatively affecting the others are highly desired.

As mentioned above, system 10 is adapted to apply a bioactive agent to a delivery substrate. As used herein, a “delivery substrate” is used to describe a medium onto which one or more bioactive agents may be applied. The delivery substrate can be coated with receiving layers such as polyvinyl alcohol, hydrogels, polytetrafluoroethylene, or other tailored biocompatible films. A delivery substrate, one or more applied bioactive agents, and other applied substances can be collectively referred to as a dosage form, which may be taken by an animal recipient. FIG. 2 schematically shows such a dosage form 12, which includes a delivery substrate 14, and an applied bioactive agent 16. It should be understood that the dosage form may also include one or more auxiliary components.

As shown in FIG. 3, a delivery substrate may be configured as a sheet 18 that includes a plurality of discrete dosage portions 20 onto which a desired amount of bioactive agent can be applied to produce a dosage form. The bioactive agent can be applied to each of the plurality of dosage portions and then the dosage portions may be separated from one another for individual delivery to one or more recipients. Sheet 18 is provided as a nonlimiting example, and doses may be applied to delivery substrates taking different forms. For example, a roll of substrate may be used for high speed production of dosage forms. It is also within the scope of this disclosure to individually apply only a single dose of a bioactive agent to a delivery substrate at a particular time instead of applying plural doses to a corresponding plurality of different dosage portions. In other words, dosage forms may be prepared one at a time or several at the same time, or at least one after the other.

A dosage form can be configured for oral delivery, topical delivery, or any other suitable delivery mode. When configured for oral delivery, a dosage form may be configured to be ingested or the dosage form may be configured to be removed from the oral cavity after the bioactive agent is released. When configured for ingestion, the delivery substrate can be configured to dissolve or degrade in body fluids and/or enzymes, or the delivery substrate can be made of non-degradable materials that are readily eliminated by the body. The delivery substrate may be hydrophilic and readily disintegrate in water. Furthermore, the delivery substrate may be configured so that dissolution or disintegration is enabled, or enhanced, at the pH of the fluids in the stomach or upper intestine.

Materials used to construct a delivery substrate may be selected to improve the final dosage form. For example, the substrate properties can be tailored to receive the impinging drops in an optimized fashion and to release corresponding solvents as required. The delivery substrate may be configured to minimize unintended interactions with the bioactive agent dispensed on the delivery substrate. The delivery substrate may also be configured to remain stable over extended periods of time, at elevated temperatures, and at high or low levels of relative humidity. In addition, a delivery substrate can be configured to resist the growth of microorganisms. Further, a delivery substrate may be configured with reasonable mechanical properties, such as tensile strength and tear strength.

A delivery substrate may include polymeric and/or paper organic film formers. In some embodiments, inorganic films may be used. Nonlimiting examples of delivery substrates include starch (natural and chemically modified), glycerin based sheets with or without a releasable backing, and the like; proteins such as gelatin, wheat gluten, and the like; cellulose derivatives such as hydroxypropylmethylcellulose, methocel, and the like; other polysaccharides such as pectin, xanthan gum, guar gum, algin, pullulan (an extracellular water-soluble microbial polysaccharide produced by different strains of Aureobasidium pullulans), and the like; sorbitol; seaweed; synthetic polymers such as polyvinyl alcohol, polymethylvinylether (PVME), poly-(2-ethyl 2-oxazoline), polyvinylpyrrolidone, and the like. Further examples of edible delivery substrates are those that are based on milk proteins, rice paper, potato wafer sheets, and films made from restructured fruits and vegetables. It should be understood that one or more of the above listed substrate materials, as well as other substrate materials, may be used in combination in some embodiments.

Using an ingestible delivery substrate containing a water-expandable foam can facilitate the rapid release of the bioactive agent once taken by the recipient. Examples of such materials are an oxidized regenerated cellulose commercially available from Johnson and Johnson under the trademark SURGICEL®, and a porcine derived gelatin powder commercially available from Pharmacia Corporation under the trademark GELFOAM®.

As schematically shown in FIG. 1, system 10 includes a data interface 30, a control subsystem 32, a positioning subsystem 34, and a depositing subsystem 36. Systems similar to system 10 have been used for printing extremely small droplets of ink onto paper to create an image. Such systems are commonly referred to as “inkjet” printing systems. As described herein, technology used to print ink onto paper may be adapted to apply a bioactive agent to a delivery substrate. Such application systems are highly refined and can be used in high volume industrial applications and/or low volume personal applications. Highly developed printing methods can be adapted to fabricate and control drug production in a very reproducible and high speed process. Furthermore, it should be understood that advances in inkjet printing technology may be utilized to precisely apply a bioactive agent to a delivery substrate, thereby enhancing control of the dissolution rate of the bioactive agent.

Control subsystem 32 can include componentry, such as a printed circuit board, processor, memory, application specific integrated circuit, etc., which effectuates application of a bioactive agent onto the delivery substrate in accordance with received information 40. Information 40 may be received via a wired or wireless data interface 30, or other suitable mechanism. Such information may include instructions for applying a particular bioactive agent to the delivery substrate according to one or more application parameters. Upon receiving such instructions, the control subsystem can cause positioning subsystem 34 and depositing subsystem 36 to cooperate to apply a bioactive agent to a sheet 18 of delivery substrate 14, thus producing a dosage form 12 that may be taken by a recipient.

Positioning subsystem 34 can control the relative positioning of the depositing subsystem and the delivery substrate onto which the bioactive agent is applied. For example, positioning subsystem 34 can include a sheet feed that advances the delivery substrate through an application zone 42 of the depositing subsystem. The positioning subsystem can additionally or alternatively include a mechanism for laterally positioning the depositing subsystem, or a portion thereof, relative to the delivery substrate. The relative position of the delivery substrate and the depositing subsystem can be controlled so that the bioactive agent is applied onto only a desired portion of the delivery substrate.

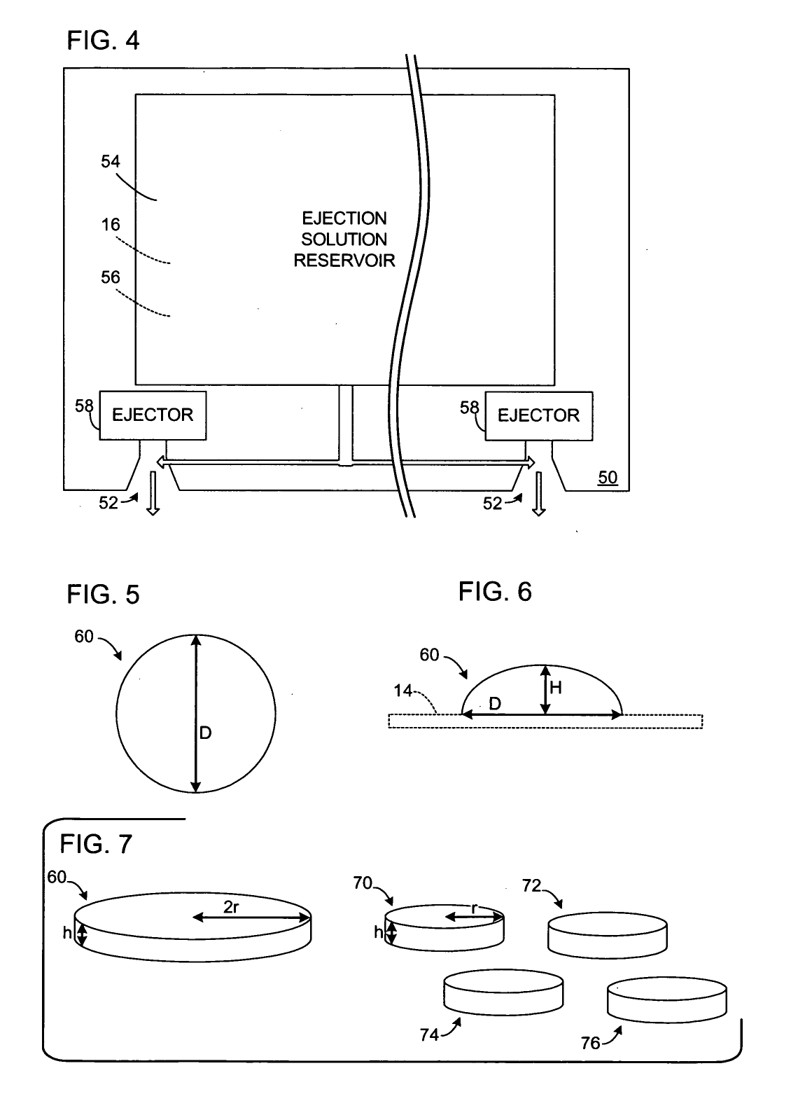

FIG. 4 schematically shows a portion of an exemplary depositing subsystem in the form of an ejection cartridge 50, which may include one or more nozzles 52 adapted to eject bioactive agent 16 onto a delivery substrate. The bioactive agent can be ejected as a constituent element of an ejection solution 54 that includes a carrier solvent 56, such as ethanol. The bioactive agent can be ejected onto the delivery substrate in the form of an ejection “drop.” The size, geometry, and other aspects of nozzle 52 can be designed to reliably eject drops having a desired volume. Current application systems can apply drops ranging from as small as nanoliters to femtoliters, and even smaller drop sizes may be possible. Each nozzle can be similarly configured so that ejected drops have approximately the same volume.

As shown in FIG. 4, a nozzle can be associated with an ejector 58, such as a semiconductor resistor, that is operatively connected to a control subsystem. Ejector 58 is designed to cause drops of ejection solution 54 to be ejected through a nozzle 52. In embodiments that utilize a resistor as an ejector, the control subsystem may activate the resistor by directing current through the resistor in one or more pulses. Each ejector can be configured to receive an ejection pulse via a conductive path that leads to the ejector. The control subsystem can route current to the individual ejectors through such conductive paths based on received instructions. Ejection pulses can be used to selectively cause the ejector to heat the ejection solution and at least partially vaporize the solution to create an ejection bubble. Expansion of the ejection bubble can cause some of the solution to be ejected out of the corresponding nozzle onto the delivery substrate. Ejection of the solution can be precisely timed to fire onto a desired portion of the delivery substrate, the relative position of which may be controlled by the positioning subsystem with great accuracy. The control subsystem can cause the various ejectors to eject the bioactive agent through the corresponding nozzles onto the desired portions of the delivery substrate in accordance with received instructions, such as instructions received in the form of an application signal.

Application of bioactive agent onto a delivery substrate in the form of ejected drops produces a “dot” of the bioactive agent on the delivery substrate. The term “dot” is used to refer to the bioactive agent drop once it contacts the delivery substrate. A dot may be in liquid or solid form. For example, a liquid drop is typically applied to the substrate, and upon contacting the substrate is referred to as a dot. The liquid dot may then dry, or otherwise settle, thus becoming a dry dot on the delivery substrate. In some embodiments, the bioactive agent in the drop will stay in a thin layer near the surface of the media. However, some media can be porous, and when the drop contacts the media the bioactive agent can spread outward and/or penetrate into the media resulting in dot gain and/or penetration. Dot gain is the ratio of the final diameter of a dot on the media to its initial diameter. Dot penetration is the depth that the drop soaks into the media. The physical and/or chemical properties of the dots can enhance dissolution rates without disrupting the permeability and specificity of the bioactive agent. Controlled dot placement, high surface-to-mass ratio of the dots, and digital mass deposition control of the dots can be used to address significant dissolution rate and dosage control issues faced by the pharmaceutical industry.

FIGS. 5 and 6 schematically show an exemplary dot 60 on a delivery substrate 14. Dot 60 has virtually no dot gain or dot penetration, as may be the case when an ejection solution is applied to a delivery substrate having a polytetrafluoroethylene, or other nonwettable, surface. Application to such a nonwettable surface is herein used for the purpose of simplicity. It should be understood that the general principals set forth in this disclosure also can apply when ejection solution is applied to a wettable delivery substrate.

Exemplary dot 60 is half of an oblate spheroid, characterized by a substantially circular horizontal cross-section having a radius R and a substantially elliptical vertical cross section having a height H. The geometric surface area (S) of dot 60 is given by the following equation:

$$S = \frac{1}{2} ( 2 π {R}^{2} + π \frac{{H}^{2}}{e} ln ( \frac{1 + e}{1 - e} ) )$$

As described in more detail below, the geometric surface area of a dot can affect attributes of the bioactive agent, such as dissolution rate of the bioactive agent. It should be understood that dot 60 is provided as a nonlimiting example, and other dot geometries are possible. The geometric surface area of such differently shaped dots can also affect attributes of the bioactive agent, such as dissolution rate of the bioactive agent.

A depositing subsystem may be adapted to apply one or more different bioactive agents, which may be carried in corresponding ejection solutions. In some embodiments, a depositing subsystem may include two or more ejection cartridges that are each configured to apply a different bioactive agent to a corresponding delivery substrate and/or eject solution having different drop volumes. Furthermore, a depositing subsystem may be configured to interchangeably receive different ejection cartridges, which are individually configured to apply different bioactive agents to corresponding delivery substrates. Interchangeable ejection cartridges may also be used to replace an empty ejection cartridge with a full ejection cartridge. It is within the scope of this disclosure to utilize other mechanisms for applying a bioactive agent onto a delivery substrate, and ejection cartridge 50 is provided as a nonlimiting example. For example, a depositing subsystem may include an ejection cartridge that utilizes an ejection-head having ejectors configured to effectuate fluid ejection via a nonthermal mechanism, such as vibrational displacement caused by a piezoelectric ejection element.

As described herein, application systems, such as system 10, can be used to prepare a dosage form that includes a bioactive agent with an accurately controlled dose, dissolution rate, and dosing profile. In particular, system 10 can be used to prepare a dosage form that has a high dissolution rate and a very accurate dose. Application systems can very accurately place small drops of ejection solution onto a delivery substrate. Ejection of bioactive agents through application devices has been demonstrated as non destructive to small and large molecule bioactive agents. The method involves no chemical modification of the bioactive agent which might affect the effectiveness of the bioactive agent or cause undesired side effects. It is similar to dissolution and reprecipitation of a drug onto a suitable substrate.

Digitally addressable application technology enables highly reproducible deposition of bioactive agents for dosage control. Application systems can actively measure drop sizes and nozzle malfunctions, and use such information to achieve an accurate dosage by correcting and/or compensating for any irregularities. Furthermore, the same dosage may be applied to a delivery substrate in virtually unlimited different dot patterns, dot sizes, dot shapes, etc. Therefore, attributes of the dosage form, such as dissolution rate, may be controlled independently of the amount of bioactive agent that makes up the dose.

The deposition characteristics of a bioactive agent on a delivery substrate can be influenced by the manner in which the bioactive agent is applied to the delivery substrate. As used herein, “deposition characteristic” is used to refer to a physical and/or chemical characteristic of a bioactive agent, as applied to a delivery substrate. The deposition characteristics can affect attributes of the bioactive agent, such as dissolution rate. Nonlimiting examples of deposition characteristics include dot size, dot geometric surface area, dot mass, dot surface-to-mass ratio, dot topography, dot topographic surface area, dot geometry, dot layering, crystal morphology, solubility, and physio- and/or chemio-interactions between the bioactive agent and the delivery substrate (e.g. covalent, ionic, hydrogen bonding). Such deposition characteristics can heavily influence the attributes of a dosage form. For example, dissolution rate is directly proportional to surface area, as demonstrated by the Noyes-Whitney Equation:

dc/dt=k*S*(Cₛ−C_{b})

Where:

dc/dt=dissolution rate

k=dissolution rate constant

S=surface area

Cₛ=saturation concentration

C_{b}=bulk solution concentration

Therefore, the ability to control deposition characteristics can provide a high level of control over the attributes of the dosage form, such as the dissolution rate of the bioactive agent on the dosage form.

A bioactive agent can be applied to a delivery substrate in a highly controlled manner. In particular, a depositing subsystem can be configured so as to eject drops having a desired size. As mentioned above, drop size can be very small, and small drop size can facilitate small dot size. Furthermore, a positioning subsystem can cooperate with a depositing subsystem to precisely place drops on a substrate. A depositing subsystem can be configured to generate a desired drop size for a particular bioactive agent. The drop size and drop pattern, as well as other characteristics of the applied bioactive agent, are highly repeatable. Therefore, dosage forms can be produced with a high degree of consistency.

Application parameters, which correspond to the manner in which the bioactive agent is applied to the delivery substrate and/or the configuration of the application system, can be set so that the bioactive agent will have desired deposition characteristics on the delivery substrate. Application parameters can be set based on a target dissolution rate, which can be achieved when the bioactive agent is applied to a delivery substrate according to the set application parameters. Nonlimiting examples of application parameters which may be set to affect deposition characteristics, and consequently dissolution rates, include nozzle size, nozzle shape, chamber size, chamber shape, pulse character, firing frequency, firing modulation, burst number (number of drops fired at a particular frequency over a particular period of time), firing energy, turn-on-energy, pulse warming, back pressure (pressure at which fluid is supplied to chamber and/or nozzle), substrate temperature, drop spacing, deposition patterns, number of passes, drying methods (ambient temperature, solution temperature, solvent vapor pressure, etc.), dry time between passes, bioactive agent concentration in the ejection solution, solution viscosity, solution surface tension, and solution density.

Application parameters can be organized into primary and secondary application parameters. Primary application parameters can be selected to determine a broad range of the drop size or composition utilized to form the dots on the delivery substrate. Non-limiting examples of primary application parameters include nozzle geometry (nozzle dimensions and shape), resistor size, firing chamber geometry, drying methods, and bioactive fluid properties. Some primary application parameters are substantially fixed, meaning that they are set before application of the bioactive agent is initiated. Primary application parameters can be specified to generally determine the coarse or approximate values for drop size and composition.

Secondary application parameters can be selected to determine a narrower range for drop size within the broader range discussed above. Non-limiting examples of secondary application parameters include fire pulse parameters (pulse shape, voltage, current, or duration), pulse warming parameters, firing frequency, back pressure, burst number, and ejector substrate temperature. Some secondary application parameters are variable, meaning that they can be selectively modified after the application system is created to modulate a drop size or other characteristics to within a tolerance.

One or more primary and/or secondary application parameters can be set to achieve a desired dot size, which can affect a deposition characteristic, including the surface-to-mass ratio of the bioactive agent on the delivery substrate. For example, the dot size of the applied bioactive agent can be kept relatively small by applying relatively small drops to a delivery substrate. Current application systems can apply drops ranging from nanoliters to femtoliters, and even smaller drop sizes may be possible. Nozzle size and chamber size are exemplary application parameters that can be set to achieve small drop sizes. The application of very small drops to a suitable delivery substrate can facilitate very high geometric surface-to-mass ratio application of the bioactive agent in a very repeatable and predictable process. The variability in drop volumes ejected from an ejection cartridge, such as a thermal ejection cartridge or a piezoelectric ejection cartridge, can be substantially less than the variability previously achievable using prior art application methods. Such drops can form substantially uniformly sized dots. The ability to consistently produce substantially uniformly sized dots can help attain a desired dissolution rate of a bioactive agent. In particular, uniformly sized dots can individually dissolve at a consistent and predictable rate, thus providing substantial control of the dissolution rate of a plurality of dots. Using current ejection cartridge manufacturing procedures, the standard deviation in drop volume may be approximately 10% to approximately 25% or less of the mean drop volume, and even smaller standard deviations are possible. In contrast, other methods of applying a pharmaceutical to a delivery media, such as aerosol spraying, may have a standard deviation of approximately 40% or greater of the mean drop volume. In particular, such methods have not been able to consistently produce a standard deviation of 15% or less, which is achievable using the systems and methods described herein. In other words, ejection of a solution through a precisely manufactured nozzle, as described herein, can be substantially more consistent and controllable than other application methods. Furthermore, consistent drop volume can facilitate consistent dot size, such as where a standard deviation for a geometric surface-to-mass ratio of the dots is less than approximately 15% of a mean geometric surface-to-mass ratio of the dots.

Dot size can also be kept relatively small by decreasing the concentration of dissolved bioactive agent in an ejection solution and/or by increasing solvent removal rates, which can be influenced by application parameters such as solvent composition (low flash point), drop size, drying temperature, and/or vapor pressure. For example, smaller drops tend to increase the removal rate of solvent due to more proportional droplet surface area, and increased temperatures (e.g. solution, ambient, and/or substrate) tend to enhance evaporation of the solvent. In some embodiments, depositing system 36 can include a heating assembly, such as an IR/convection oven, to heat up and evaporate unwanted solvents from the delivery substrate after the bioactive agent has been deposited. The ability to apply a bioactive agent with a small dot size facilitates high dissolution rates because the same amount of bioactive agent may be applied in many small dots, which have a relatively high net geometric surface area, instead of in fewer large dots, which have a relatively small net geometric surface area.

FIG. 7 schematically shows how small dot size can increase surface-to-mass ratio, and therefore increase dissolution rate. As illustrated, dot 60 has an exemplary cylindrical volume equal to V=4πr²h, and dots 70, 72, 74, and 76 each have exemplary cylindrical volumes equal to V=πr²h. Therefore, the four smaller dots have the same collective volume as the larger dot. Assuming equal densities, the smaller dots also collectively have the same mass as the larger dot. However, the larger dot has a geometric surface area equal to S=4πr(h+r), while the geometric surface area of one of the smaller dots is equal to S=πr(2h+r). Therefore, the net geometric surface area of the four smaller dots combined is equal to S=4πr(2h+r). As can be seen, assuming cylindrical geometry, the surface area of the 4 smaller dots will be larger than the surface area of the larger dot if the heights of the dots do not equal zero. The above example shows dots that have cylindrical geometries for the purpose of simplicity. However, it should be understood that substantially more complicated drop geometries are possible, and small relative dot size can improve the net geometric surface-to-mass ratio for such geometries.

The deposition pattern of drops applied to the delivery substrate is another nonlimiting example of an application parameter that may be used to affect a deposition characteristic, including the surface-to-mass ratio, of the bioactive agent on the delivery substrate. In particular, the surface-to-mass ratio can be controlled by selecting the spacing between adjacent drops. Sufficient spacing between adjacent drops can prevent adjacent dots from coalescing, which tends to decrease the geometric surface-to-mass ratio. Conversely, drops may be applied sufficiently close to one another to effectively build up the bioactive agent so as to have a lower geometric surface-to-mass ratio than would be present in separated dots having the same net mass. The same amount of a bioactive agent may be applied with different dot spacing, which can correspond to different surface-to-mass ratios, thereby permitting customized deposition characteristics for the bioactive agent. Application systems can precisely place drops, such as consistently within at least approximately 1×10⁻⁵ meters (10 microns) of an intended target on the delivery substrate. Such precise placement facilitates highly reproducible dot patterns.

Drop placement, or more precisely, drop precision of approximately 1×10⁻⁵ meters is sufficient for an application system to precisely place about 2400 discrete drops per inch. A 2400 drops per inch application system can produce a dot to dot spacing of approximately 11 microns. More precise drop placement is possible by setting one or more parameters to achieve improved placement accuracy. For example, a nozzle can be designed with a long bore to achieve greater precision. Sustained precision can be maintained by frequently cleaning nozzles of the depositing subsystem, thereby reducing drool or crud that may puddle around the nozzle and thereby affect ejection accuracy. Precise drop placement may also be influenced by controlling drop firing velocity (speed and direction). Furthermore decreasing nozzle-to-media distance can reduce the effect of drop speed variability on drop precision by minimizing the area in which drops may land. Drops can decelerate between the nozzle and the media due to factors such as air resistance. Smaller drop volumes can correspond to faster deceleration rates due to less drop momentum. When a drop is fired at a speed higher than average, it can land on the media slightly before a targeted location. Conversely, when a drop is fired at a slower than average speed, it can land after a targeted location. Furthermore, variability introduced in drop trajectory and/or the relative speed between the media and the nozzles can be exaggerated over longer drop firing distances. Therefore, decreasing nozzle-to-media distance can help reduce some variability that could limit drop precision. However, some types of media may swell, and nozzles can be spaced sufficiently to avoid crashing the media. A nozzle-to-media distance of approximately 0.5 to 1.3 millimeters has been found to provide adequate spacing while limiting drop placement variability to an acceptable level. Control of the above described exemplary parameters enables drops to be very precisely placed compared to other known application methods.

Drops can be placed so that they are spaced apart from each other or drops can be purposefully placed at least partially on top of one another. In either case, each ejected drop can be precisely placed in a desired location. Drop placement does not have to be left to random chance, as may be the case using other application methods, such as aerosol spray delivery. Precise drop placement can be used to effectuate a desired dot pattern or dot spacing. The relative spacing of two or more adjacent drops can change the surface-to-mass ratio of applied dots, and therefore control the dissolution rate of the applied bioactive agent.

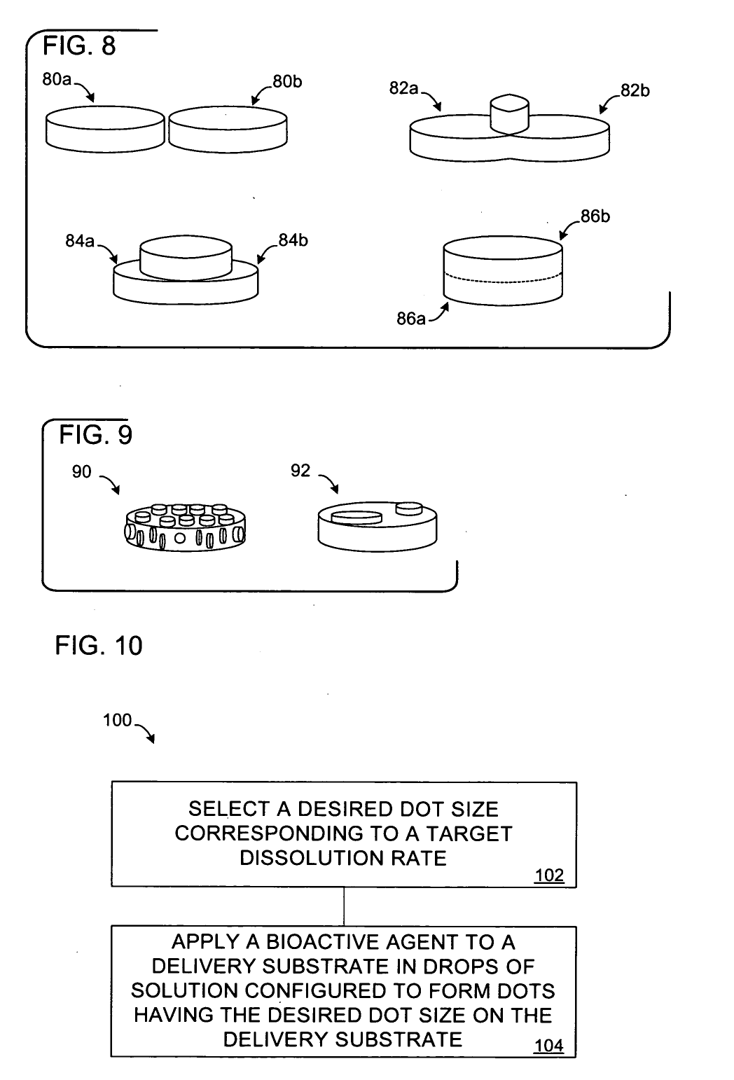

For example, FIG. 8 schematically shows four alternate dot patterns corresponding to four different surface-to-mass ratios. Dots 80a and 80b are spaced apart from one another, and do not overlap. Dots 82a and 82b are spaced closer together, and slightly overlap. Dots 84a and 84b are spaced even closer together, and there is considerable overlap between the two dots. Finally, dots 86a and 86b are spaced one on top of the other, completely overlapping. In general, surface-to-mass ratio will decrease as the amount of dot overlap increases. Therefore, dots 80a and 80b have the highest collective surface-to-mass ratio, while dots 86a and 86b have the lowest collective surface to mass ratio. As described above, dissolution rate relates to the surface-to-mass ratio. Therefore, dot spacing can be selected to achieve a desired dissolution rate.

Although described in the context of two dots, it should be understood that spacing between three or more dots may be selected to further achieve a desired dissolution rate. The spacing between all applied dots may be substantially the same for all dots, or the dots may be arranged in a pattern in which the spacing varies, such as in a repeating pattern. In either case, a high level of control over drop placement enables drops to be applied so that a standard deviation of distance between adjacent dots is less than approximately 15% of a mean distance between adjacent dots. As used in this context, adjacent dots means pairs of dots that are intended to have the same spacing as other pairs of dots. Dots that are purposefully spaced at a different distance are not considered adjacent in this context. As mentioned above, some dots can be purposefully overlapped. A high level of control over drop placement enables drops to be applied so that a standard deviation of combined geometric surface area of overlapping dots is less than approximately 15% of a mean combined geometric surface area of overlapping dots.

Dots having different sizes (corresponding to drops with different sizes, for example), may be precisely positioned to achieve a desired dissolution rate. It should be understood that FIG. 8 schematically represents dots as cylinders, and that actual dot geometry can be considerably more complex. Nonetheless, the ability to precisely control drop placement, and therefore dot pattern, can be used to control the relative dissolution rate for virtually any dot geometry.

Dot shape and/or topography are also deposition characteristics which can be influenced by application parameters. As used herein, dot shape refers to the general shape of a dot without reference to surface detail, and dot topography is used to refer to surface detail of the dot. Dot shape and/or topography can have a great effect on the topographic surface area of a dot. A highly textured surface can provide much more surface area than a smooth surface. The amount of topographic surface area typically directly corresponds to the probability that the dot will dissolve. In other words, a dot exposed on many sides, and therefore having less three-dimensional crystal lattice stabilization, is more likely to readily dissolve than a dot with less exposure and more stabilization. Application parameters that can be set to affect topographical surface area based on shape and/or topography include bioactive agent concentration in the ejection solution, and those parameters affecting drop size and solvent removal rates.

Dot topography and/or dot shape can be influenced by the crystal morphology of a dot. Some bioactive agents have many crystal forms. A noncrystalline (amorphous) form of a bioactive agent may be the fastest dissolving but can also be the most unstable and difficult to consistently reproduce, store, and deliver. Suitable amorphous forms can typically be formed by co-drying the bioactive agent with an excipient, including, but not limited to, polymer film forming agents such as pullalin, polyvinyl pyrrolidine, hydroxypropyl methyl cellulose, polyethylene glycol, and the like. Some hydrates and solvates can be more or less stable than the pure crystal forms and water can be absorbed or desorbed during storage. Different crystal morphologies can be achieved by adjusting application parameters such as solvent formulation, drop size, removal rates, and crystal templates. Crystal formation kinetics can drive a crystal form to different structures or mixtures of structures. The desired state can be selected to optimize dissolution rate while retaining adequate stability.

Desired amorphous or crystal forms can be reliably produced and stabilized because of the ability of an application system to precisely place precisely controlled solution formulations as consistently sized drops in a desired pattern, while having a high level of control over how the solution dries and/or other application parameters that may affect crystal morphology and/or dot topography. In other words, the crystal morphology and/or dot topography of each of a plurality of dots applied to a delivery substrate may be characterized by a standard deviation of topographical surface area that is less than approximately 15% of a mean topographical surface area of all such dots applied to the delivery substrate.

Bioactive agent application, as disclosed herein, may drive and control kinetic versus equilibrium phenomena more reproducibly and/or consistently than bulk processes. The kinetics and/or solvent removal may be tightly controlled by selection of appropriate application parameters, such as drop size, drop pattern, solution formulation, vapor pressure, temperature, etc. Because individual drops of solution containing the bioactive agent can be discretely applied to a delivery substrate, there is less risk of an undesired crystal form driving crystallization of an entire batch to an undesired structure (i.e. experiencing a template affect). Furthermore, application of small drops onto a delivery substrate can minimize equilibrium affects because the kinetics associated with such application methods are very fast.

FIG. 9 schematically shows two dots having different topographical surface areas. In particular, dot 90 is characterized by a highly irregular topography, as may be present in certain crystalline forms. In some embodiments, a highly irregular topography may result from small drop size and/or fast solvent removal rates. Dot 92 has a relatively smooth topography compared to dot 90. Therefore, assuming other deposition characteristics of the dots are substantially similar, dot 90 can have a faster dissolution rate than dot 92. It should be understood that dot 90 and dot 92 are illustrated in very schematic form. The actual topography of a dot can be highly variable depending on the bioactive agent forming the dot, the delivery substrate, and/or other application parameters.

Delivery substrate selection is yet another application parameter which can be set to influence deposition characteristics. For example, a delivery substrate may be selected so that the applied bioactive agent is encapsulated or entrained in interstitial spaces of the substrate, or delivery substrates may be selected so that such spaces are not available for the bioactive agent to engage. When a bioactive agent is at least partially encapsulated, relatively less surface area of the bioactive agent will be exposed, and therefore dissolution rate may be decreased. Therefore, a relatively porous substrate may be selected when slower dissolution rates are desired. Relatively high dissolution rates may also be facilitated by delivery substrates that are configured to minimize agglomeration by capturing the dots on or within the receiving substrate, though not necessarily encapsulating the dot.

A desired dissolution rate can be discovered through experimentation, in which one or more application parameters are varied until a desired dissolution rate is achieved. For example, parameters affecting drop size, such as nozzle size and/or chamber size, can be varied. Furthermore, additional or alternative parameters, such as solution concentration, drop pattern, and/or drying temperatures can be varied. Test dosage forms can be formed according to the set parameters. Such dosage forms can be made with different parameter settings until a desired dissolution rate is achieved. Once a desired dissolution rate is achieved, the parameters used to make the test dosage form can be used to repeatedly make dosage forms with a consistent dissolution rate.

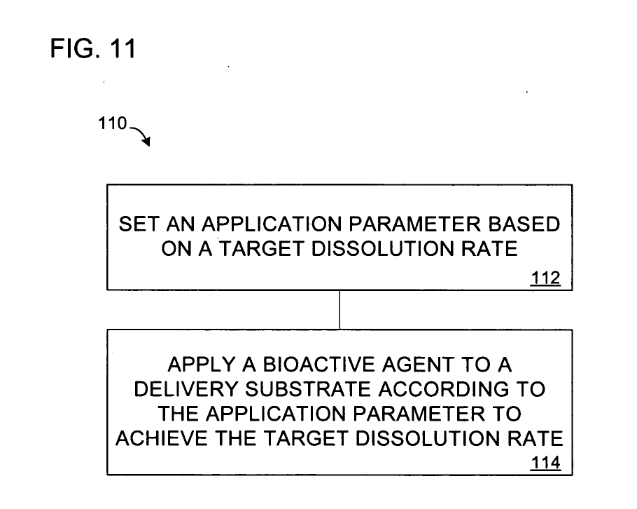

FIG. 10 is a flow chart showing an exemplary method, shown generally at 100, of controlling a dissolution rate of a bioactive agent. Method 100 includes, at 102, selecting a desired dot size corresponding to a target dissolution rate. The method further includes, at 104, applying a bioactive agent to a delivery substrate in drops of solution configured to form dots having the desired dot size on the delivery substrate. FIG. 11 is a flow chart showing another exemplary method, shown generally at 110, of controlling a dissolution rate of a bioactive agent. Method 110 includes, at 112, setting an application parameter based on a target dissolution rate. The method further includes, at 114, applying a bioactive agent to a delivery substrate according to the application parameter to achieve the target dissolution rate. Using such methods, a dosage form can be produced to have a target dissolution rate, or at least a dissolution rate substantially close to the target dissolution rate.

Although the present disclosure has been provided with reference to the foregoing operational principles and embodiments, it will be apparent to those skilled in the art that various changes in form and detail may be made without departing from the spirit and scope defined in the appended claims. The present disclosure is intended to embrace all such alternatives, modifications and variances. Where the disclosure or claims recite “a,” “a first,” or “another” element, or the equivalent thereof, they should be interpreted to include one or more such elements, neither requiring nor excluding two or more such elements.

What is claimed is:

1. A method of controlling a dissolution rate of a bioactive agent, the method comprising:
selecting a target dissolution rate;
applying a bioactive agent to a delivery substrate as a plurality of substantially uniformly sized dots to attain the selected target dissolution rate.

2. The method of claim 1, wherein applying the bioactive agent to the delivery substrate includes heating a solution including the bioactive agent with a thermal ejection element.

3. The method of claim 1, wherein applying the bioactive agent to the delivery substrate includes displacing a solution including the bioactive agent with a piezoelectric ejection element.

4. A method of controlling a dissolution rate of a bioactive agent, the method comprising:
selecting a desired dot size corresponding to a target dissolution rate;
applying a bioactive agent to a delivery substrate in drops of solution configured to form dots having the desired dot size on the delivery substrate.

5. The method of claim 4, wherein a volume of each of the drops is less than approximately 1×10⁻⁹ liters.

6. The method of claim 4, wherein a volume of each of the drops is less than approximately 1×10⁻¹¹ liters.

7. The method of claim 4, wherein a volume of each of the drops is in the range of approximately 10×10⁻¹² liters and 70×10⁻¹² liters.

8. The method of claim 4, wherein the dots are substantially uniformly sized.

9. The method of claim 4, wherein a standard deviation of drop volume is less than approximately 15% of a mean drop volume.

10. The method of claim 4, further comprising selecting a second desired dot size corresponding to the target dissolution rate; and
applying the bioactive agent to the delivery substrate in drops of solution configured to form dots having the second desired dot size on the delivery substrate.

11. The method of claim 10, wherein a standard deviation of drop volume of drops used to form dots having the second desired dot size is less than approximately 15% of a mean drop volume of drops used to form dots having the second desired dot size.

12. The method of claim 4, wherein applying the bioactive agent to the delivery substrate includes heating the solution including the bioactive agent with a thermal ejection element.

13. The method of claim 12, wherein the heated solution is applied via at least two nozzles sized to eject drops of solution having substantially the same volume.

14. The method of claim 12, wherein the heated solution is applied via at least a first nozzle set and a second nozzle set, wherein the first nozzle set includes a first plurality of nozzles configured to eject drops of solution having a first volume, and wherein the second nozzle set includes a second plurality of nozzles configured to eject drops of solution having a second volume different than the first volume.

15. The method of claim 4, wherein applying the bioactive agent to the delivery substrate includes displacing the solution including the bioactive agent with a piezoelectric ejection element.

16. The method of claim 15, wherein the displaced solution is applied via at least two nozzles sized to eject drops of solution having substantially the same volume.

17. The method of claim 16, wherein the displaced solution is applied via at least a first nozzle set and a second nozzle set, wherein the first nozzle set includes a first plurality of nozzles configured to eject drops of solution having a first volume, and wherein the second nozzle set includes a second plurality of nozzles configured to eject drops of solution having a second volume different than the first volume.

18. The method of claim 4, wherein a concentration of the bioactive agent in the solution is set to form dots having the desired dot size on the delivery substrate.

19. A bioactive dosage form, comprising:
a delivery substrate; and
a plurality of dots of bioactive agent on the delivery substrate sized to achieve a target dissolution rate.

20. The bioactive dosage form of claim 19, wherein a standard deviation for a geometric surface-to-mass ratio of the dots is less than approximately 15% of a mean geometric surface-to-mass ratio of the dots.

21. The bioactive dosage form of claim 19, wherein the plurality of dots include a first set of dots and at least a second set of dots, wherein a standard deviation for a geometric surface-to-mass ratio of the first set of dots is less than approximately 15% of a mean geometric surface-to-mass ratio of the first set of dots, and wherein a standard deviation for a geometric surface-to-mass ratio of the second set of dots is less than approximately 15% of a mean geometric surface-to-mass ratio of the second set of dots.

22. The bioactive dosage form of claim 19, wherein the plurality of dots include dried bioactive agent deposited as a constituent element of an ejection solution.

23. The bioactive dosage form of claim 19, wherein the delivery substrate includes an ingestible media.

24. The bioactive dosage form of claim 19, wherein the delivery substrate includes at least one of starch, glycerin, gelatin, wheat gluten, hydroxypropylmethylcellulose, methocel, pectin, xanthan gum, guar gum, algin, pullulan, sorbitol, seaweed, polyvinyl alcohol, polymethylvinylether, poly-(2-ethyl 2-oxazoline), polyvinylpyrrolidone, milk proteins, rice paper, potato wafer, and films made from restructured fruits and vegetables.

25. The bioactive dosage form of claim 19, wherein the delivery substrate includes pullulan.

26. A bioactive agent application system, comprising:
a nozzle;
an ejector associated with the nozzle, wherein the nozzle and associated ejector are collectively configured to selectively eject the bioactive agent in drops of solution configured to form dots having a dot size corresponding to a target dissolution rate.

27. The bioactive agent application system of claim 26, wherein the nozzle and associated ejector are one of a plurality of nozzle and associated ejector pairs, each configured to form dots having a dot size corresponding to a target dissolution rate.

28. A bioactive agent application system of claim 27, wherein a standard deviation for a geometric surface-to-mass ratio of the dots formed from the ejected drops is less than approximately 15% of a mean geometric surface-to-mass ratio of the dots.

29. The bioactive agent application system of claim 26, wherein at least one nozzle and associated ejector pair is configured to form dots having a different dot size than dots formed by at least one other nozzle and associated ejector pair.

30. A bioactive agent application system of claim 26, wherein a volume of each ejected drop is less than approximately 1×10-11 liters.

31. A bioactive agent application system of claim 26, wherein a standard deviation of drop volume is less than approximately 15% of a mean drop volume.

32. A method of controlling a dissolution rate of a bioactive agent, the method comprising:
setting an application parameter based on a target dissolution rate; and
applying a bioactive agent to a delivery substrate according to the application parameter to achieve the target dissolution rate.

33. The method of claim 32, wherein applying the bioactive agent to the delivery substrate includes ejecting an ejection solution including the bioactive agent onto the delivery substrate as a plurality of drops.

34. The method of claim 33, wherein each of the plurality of drops is sized to achieve the target dissolution rate.

35. The method of claim 34, wherein a volume of each of the plurality of drops is less than approximately 1×10⁻¹¹ liters.

36. The method of claim 34, wherein a standard deviation of drop volume for the plurality of drops is less than approximately 15% of a mean drop volume of the plurality of drops.

37. The method of claim 32, wherein applying the bioactive agent to the delivery substrate includes heating an ejection solution including the bioactive agent with a thermal ejection element.

38. The method of claim 32, wherein applying the bioactive agent to the delivery substrate includes displacing an ejection solution including the bioactive agent with a piezoelectric ejection element.

39. The method of claim 32, wherein the bioactive agent is applied to the delivery substrate in an ejection solution including the bioactive agent and a carrier solvent.

40. The method of claim 32, wherein the application parameter is selected to effectuate a deposition characteristic of the bioactive agent on the delivery substrate, wherein the deposition characteristic affects the dissolution rate of the bioactive agent.

41. The method of claim 40, wherein the deposition characteristic is dot size.

42. The method of claim 40, wherein the deposition characteristic is dot geometric surface-to-mass ratio.

43. The method of claim 32, wherein the application parameter is one of a plurality of application parameters that collectively affect the dissolution rate of the bioactive agent.

44. The method of claim 32, wherein the application parameter includes nozzle size.

45. The method of claim 32, wherein the application parameter includes chamber size.

46. The method of claim 32, wherein the application parameter includes at least one of nozzle size, nozzle shape, chamber size, chamber shape, pulse character, firing frequency, firing modulation, burst number, firing energy, turn-on-energy, pulse warming, back pressure, substrate temperature, drop spacing, deposition pattern, number of passes, drying methods, dry time between passes, bioactive agent concentration in the ejection solution, solution viscosity, solution surface tension, and solution density.

47. A bioactive dosage form, comprising:
a delivery substrate; and
a bioactive agent that is applied to the delivery substrate according to an application parameter selected to achieve a target dissolution rate.

48. A bioactive agent application system, comprising:
a depositing subsystem configured to fire a plurality of drops including a bioactive agent onto a delivery substrate according to at least one application parameter selected to achieve a target dissolution rate.
